(19) United States
(12) Patent Application Publication
(10) Pub. No.: US 2007/0196347 A1
(43) Pub. Date: Aug. 23, 2007

(54) Methods for preventing neurological events

(76) Inventors: Anthony Kam Chuen Chan, Ancaster (CA); Jeffrey I. Weitz, Ancaster (CA); Jack Hirsh, Burlington (CA); Paul Tressel, Hamilton (CA); Roy Berry, Burlington (CA)

Correspondence Address: MCCARTHY TETRAULT LLP, BOX 48, SUITE 4700,, 66WELLINGTON STREET WEST, TORONTO, ON, M5K 1E6, CA

(21) Appl. No.: 10/567,737

(22) PCT Filed: Aug. 12, 2004
(86) PCT No.: PCT/CA04/01497

§ 371 Date: Dec. 29, 2006

Related U.S. Application Data
(60) Provisional application No. 60/494,120, filed on Aug. 12, 2003.

(51) Int. Cl. A61K 38/55; A61K 31/727

(52) U.S. Cl. 424/94.1; 514/54; 514/56

(57) ABSTRACT

The present invention relates to therapeutic methods for preventing or reducing neurological events utilizing a glycosaminoglycan and a serpin, including complexes and conjugates thereof. Methods of the present invention may be advantageous for preventing or reducing neurological events prior to, or during medical or surgical procedures, and after a neurological event. In particular, the present invention deals with neurological events associated with the generation of emboli that can lodge in the brain and/or cerebral circulation during cardiac surgery.

### FIELD OF THE INVENTION

The invention relates generally to preventing or reducing neurological events prior to, during, or following medical or surgical procedures.

### BACKGROUND OF THE INVENTION

There have been important advances in cardiac surgery in the last decades including procedures such as coronary artery bypass grafting (CABG) and cardiac repair or replacement surgery. Cardiopulmonary bypass (CPB) is generally used in these procedures to divert blood through an extracorporeal circuit to allow for the patient's heart and lungs to be stilled during surgery.

There has been growing awareness of the adverse neurological effects associated with cardiac surgery, and in particular CPB. Approximately 500,000 people each year in the United States alone undergo cardiovascular surgical procedures that use cardiopulmonary bypass (CPB) and neurocognitive deficits have been reported to occur in over 50% of these patients (Newman et al. “Longitudinal Assessment of Neurocognitive Function After Coronary-Artery Bypass Surgery,” New England Journal of Medicine 2001; 344: 395-402). The reported incidence, as measured by neuropsychological testing, ranges from 40-61% within the first week following surgery (Gugino L D et al, 1999; Vingerhoets, G. et al, 1997; Rodig G. et al, 1999; Newman M F, et al, 2001; Grigore A M et al, 2002; and Llinase R et al, 2000). Although there is substantial resolution in neurocognitive dysfunction within 6 weeks to 6 months, up to 35% of patients have neurocognitive defects that persist for at least a year (Di Carlo, A et al, 2001). Neurocognitive deficits also occur with other forms of surgery (Vingerhoets, 0, et al 1997; Van Dijk, D. et al, 2002), but the incidence of neurocognitive deficits is highest after CPB.

The etiology of neurocognitive dysfunction is thought to be the result of microemboli. Several studies have been performed using Transcranial Doppler to detect microemboli as high-intensity transient signals (HITS) during cardiac surgery (Di Carlo A et al, 2001 and Jacobs, A. et al, 1998). In these studies, HITS were associated with neurocognitive deficits, especially with respect to memory loss. Possible sources of the microemboli include air, thrombi, and fat from cellular or particulate matter promoted by the bypass pump (Jacobs A et al, 1998). Of these, thromboemboli are thought to be most important.

Currently, unfractionated heparin (UFH) is the standard agent used for anticoagulation during cardiopulmonary bypass (CPB). Although UFH provides sufficient anticoagulation to prevent clotting within the bypass circuit its inability to inactivate fibrin-bound thrombin (Hogg P J and Jackson Cm, 1990, Weitz J I et al, 1998 and Weitz, J I et al, 1990) as well as its tendency to activate platelets (Xiao Z et al, 1998) limits the ability of UFH to reduce thrombin generation and the development of thromboemboli during CPB.

It would be beneficial to eliminate or reduce the potential of neurological events associated with medical and surgical procedures. In particular, there is a need for substances that reduce or eliminate cardiac embolization.

The citation of any reference herein is not an admission that such reference is available as prior art to the instant invention.

### SUMMARY OF THE INVENTION

The present invention relates to therapeutic methods for preventing or reducing neurological events utilizing a glycosaminoglycan and a serpin. Methods of the present invention may be advantageous for protecting or reducing neurological events prior to or during medical or surgical procedures, and after a neurological event.

In particular, the present invention deals with neurological events associated with the generation of emboli (in particular thromboemboli) that can lodge in the brain and/or cerebral circulation (i.e. cardiac embolization) during surgery, in particular cardiac surgery. Neurological events resulting from embolization contribute to problems including stroke, lengthy hospital stays, and in some instances death.

An aspect of the invention relates to a therapeutic application of a glycosaminoglycan and a serpin, or conjugates or complexes thereof, to provide protection to a subject against neurological events, or reduce such neurological events.

In an aspect the invention provides a method of preventing or reducing neurological events in a subject comprising administering a therapeutically effective dosage of a glycosaminoglycan and a serpin, or conjugates or complexes thereof, to the subject to prevent or reduce the neurological events.

In another aspect, the present invention relates to a therapeutic application of a glycosaminoglycan and a serpin, or conjugates or complexes thereof, to provide protection to an individual's central nervous system, in particular an individual's brain, prior to scheduled, or unscheduled, procedures that may affect the central nervous system, in particular, the brain.

In an embodiment, the invention provides a method for reducing emboli (in particular, thromboemboli) in the cerebral circulation in a subject comprising administering an amount of a glycosaminoglycan and a serpin, or conjugates or complexes thereof, effective to reduce the emboli.

The invention also relates to a method of cerebral embolic protection in a subject comprising administering an amount of a glycosaminoglycan and a serpin, or conjugates or complexes thereof, to prevent or reduce emboli in the cerebral circulation.

The invention relates to a method for protecting a subject against cerebral embolization comprising administering an amount of a glycosaminoglycan and a serpin, or conjugates or complexes thereof, that prevents or reduces the amount of emboli that reach the cerebral vasculature.

The invention also provides methods for eliminating or minimizing cerebral embolization during invasive cardiac procedures in a subject comprising administering a therapeutically effective amount of a glycosaminoglycan and a serpin, or conjugates or complexes thereof.

Further, the invention provides a method of preventing or reducing emboli from a bypassed heart region prior to removal of the region from bypass comprising administering an amount of a glycosaminoglycan and a serpin, in particular conjugates or complexes thereof, to prevent or reduce the emboli.

The invention also provides a method for improving the outcome of cardiac surgery in a subject undergoing cardiopulmonary bypass surgery comprising administering a therapeutically effective amount of a glycosaminoglycan and a serpin, or conjugates or complexes thereof.

The invention also provides a method of performing cardiac surgery, in particular, CABG surgery, in which a therapeutically effective amount of a glycosaminoglycan and a serpin, or conjugates or complexes thereof, are administered peri-operatively to a subject undergoing cardiopulmonary bypass to reduce the effects of emboli.

In an aspect of the invention the glycosaminoglycan and serpin provide synergistic activity in preventing or reducing neurological events. In another aspect a method of preventing or reducing cerebral emboli in a subject is provided comprising administering to a subject in need thereof, synergistically effective amounts of a glycosaminoglycan and a serpin.

The present invention also provides compositions comprising a combination of a therapeutically effective amount of a glycosaminoglycan and a serpin together with a pharmaceutically acceptable excipient, carrier, or vehicle. The present invention also contemplates a pharmaceutical composition in separate containers and intended for simultaneous or sequential administration, comprising a glycosaminoglycan and a serpin, both together with pharmaceutically acceptable excipients, carriers, or vehicles.

In accordance with one aspect, a pharmaceutical composition is provided comprising a glycosaminoglycan and a serpin effective to exert a synergistic effect to prevent or reduce neurological events, in particular a neurological event associated with emboli more particularly, cerebral embolization. The method also provides pharmaceutical compositions comprising a synergistically effective amount of a combination of a glycosaminoglycan and a serpin in a pharmaceutically acceptable excipient, carrier, or vehicle.

In another aspect the invention relates to a method of using a glycosaminoglycan and a serpin, in particular conjugates or complexes thereof, in the preparation of a medicament to prevent or reduce neurological events, in particular neurological events associated with emboli, more particularly cerebral embolization.

In another aspect the invention relates to a method of using synergistically effective amounts of a glycosaminoglycan and a serpin in the preparation of a pharmaceutical composition for preventing or reducing neurological events, in particular neurological events associated with emboli, more particularly cerebral embolization.

These and other aspects, features, and advantages of the present invention should be apparent to those skilled in the art from the following drawings and detailed description.

### DESCRIPTION OF THE DRAWINGS

The invention will be better understood with reference to the drawings in which:

FIG. 1 shows a timeline for a pig CPB model. Periodic blood samples and as needed (2 ml EDTA samples for CBC, 5 ml citrate plasma samples for anticoagulant assays/TATs & D-dimer, 3 ml samples for ACT, 1 ml samples in heparinized syringe for blood gas

FIG. 2 shows a diagram of a pig CPB model.

FIG. 3 are graphs of the average microemboli HITS per hour during hypothermic CPB (3A); average microemboli HITS per hour pre hypothermic CPB (3B); and average microemboli HITS per hour post hypothermic CPB (3C).

FIG. 4 is a graph showing hypothermic CPB bleeding for the identified agents expressed as ml/hour.

FIG. 5 are graphs showing protein deposition on the CPB circuit measured either as total protein (5A) or as hemoglobin (5B).

FIG. 6 are graphs showing the time course of activated clotting time during CPB for UFH(H), ATH, or AT+H and the effects of protamine sulfate.

FIG. 7 is a graph showing protamine sulfate reversal of effects of UFH(H), ATH and AT+H or Mean ACT values. ACT was measured before (pre) and during CPB and after protamine sulfate administration after CPB (Post).

FIG. 8 are graphs showing the increase of thrombin antithrombin complexes (TAT) during CPB and thereafter in the pig model.

FIG. 9 are graphs showing the levels of D-dimers during CPB and thereafter in the pig model.

FIG. 10 is a graph showing thrombi in brain sections from pigs treated with H300, ATH(3 mg), and ATH(6 mg).

FIG. 11 is a graph showing ultrasound HITS during CPB.

### DETAILED DESCRIPTION OF THE INVENTION

Glossary

Unless defined otherwise, all technical and scientific terms used herein have the same meaning as commonly understood by one of ordinary skill in the art to which this invention belongs.

Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.” The term “about” means plus or minus 0.1 to 50%, 5-50%, or 10-40%, preferably 10-20%, more preferably 10% or 15%, of the number to which reference is being made. Further, it is to be understood that “a,” “an,” and “the” include plural referents unless the content clearly dictates otherwise. Thus, for example, reference to a composition comprising “a compound” includes a mixture of two or more compounds.

“Serpin” refers to a serine protease inhibitor and is exemplified by species including but not limited to antithrombin III and heparin cofactor II. The term includes a serpin derivative. “Serpin derivative” refers to a serpin that possesses a biological activity (either functional or structural) that is substantially similar to the biological activity of a serpin. The term “derivative” is intended to include “variants” “analogs” or “chemical derivatives” of a serpin. The term “variant” is meant to refer to a molecule substantially similar in structure and function to a serpin or a part thereof. A molecule is “substantially similar” to a serpin if both molecules have substantially similar structures or if both molecules possess similar biological activity. The term “analog” refers to a molecule substantially similar in function to a serpin molecule. The term “chemical derivative” describes a molecule that contains additional chemical moieties that are not normally a part of the base molecule. A serpin may be obtained from natural or non-natural sources (e.g. recombinant or transgenic) and it may be obtained from commercial sources.

In aspects of the invention, the serpin is antithrombin III which may be plasma derived (see for example, U.S. Pat. No. 4,087,415), transgenic (see for example, U.S. Pat. No. 6,441,145), or recombinant (see for example, U.S. Pat. No. 4,632,981). In selected embodiments of the invention the serpin is recombinant or transgenic antithrombin III from GTC Biotherapeutics (Framingham, Mass.).

The term “glycosaminoglycan” refers to linear chains of largely repeating disaccharide units containing a hexosamine and uronic acid. The precise identity of the hexosamine and uronic acid may vary widely. The disaccharide may be optionally modified by alkylation, acylation, sulfonation (O— or N-sulfated), sulfonylation, phosphorylation, phosphonylation and the like. The degree of such modification can vary and may be on a hydroxyl group or an amino group. Most usually the C6 hydroxyl and the C2 amino are sulfated. The length of the chain may vary and the glycosaminoglycan may have a molecular weight of greater than 20,000 daltons, typically up to 100,000 daltons, and more typically less than 50,000 daltons. Glycosaminoglycans are typically found as mucopolysaccharides. Representative examples of glycosaminoglycans include, heparin, low molecular weight heparin, dermatan sulfate, heparan sulfate, chondroitin-6-sulfate, chondroitin-4-sulfate, keratan sulfate, chondroitin, hyaluronic acid, polymers containing N-acetyl monosaccharides (such as N-acetyl neuraminic acid, N-acetyl glucosamine, N-acetyl galactosamine, and N-acetyl muramic acid) and the like and gums such as gum arabic, gum Tragacanth and the like. [See Heinegard, D. and Sommarin Y. (1987) Methods in Enzymology 144:319-373.]

In aspects of the invention, the glycosaminoglycan is heparin or low molecular weight heparin. In an embodiment, the glycosaminoglycan is heparin having a molecular weight in the range 6,000 to 30,000.

The term “pentasaccharide” or “pentasaccharide sequence” refers to a key structural unit of heparin that consists of three D-glucosamine and two uronic acid residues. The central D-glucosamine residue contains a unique 3-O-sulfate moiety. The pentasaccharide sequence represents the minimum structure of heparin that has high affinity for antithrombin (Choay, J. et al., Biochem Biophys Res Comm 1983; 116: 492-499).

In an embodiment, the glycosaminoglycan is a “high affinity” heparin enriched for species containing one copy or more than one copy of the pentasaccharide sequence.

In embodiments of the invention the glycosaminoglycan is a commercially available heparin or low molecular weight heparin including without limitation Lovenox™ (Aventis), Fragmin™ (Pfizer), Innohep™ (Pharmion), Clivarine™ (Abbott), Arixtra (Pondaprinux) (Sanofi) or derivatives thereof.

The invention also contemplates the use of conjugates or complexes comprising a serpin associated with a glycosaminoglycan. The term “associate”, “association” or “associating” refers to a condition of proximity between a group of a glycosaminoglycan and a serpin or serpin derivative, or parts or fragments thereof. The association may be non-covalent i.e. where the juxtaposition is energetically favored by for example, hydrogen-bonding, van der Waals, or electrostatic or hydrophobic interactions, or it may be covalent.

Selected methods of the present invention use an antithrombin and heparin covalent conjugate (i.e. ATH) as described in U.S. Pat. Nos. 6,491,965 and 6,562,781, Klement et al. Biomaterials 23:527-535, 2002 and in Berry L., Andrew M. and Chan A. K. C. Antithrombin-Heparin Complexes (Chapter 25). In: Polymeric Biomaterials. Part II: Medical and Pharmaceutical Applications of Polymers. (Second Edition) Ed. S. Dumitriu. Marcel Dekker Inc., New York, pp. 669-702, 2001, and in copending U.S. application Ser. No. 60/448,116 filed Feb. 20, 2003, which are incorporated herein in their entirety by reference. The antithrombin in ATH may be derived from plasma (see for example, U.S. Pat. No. 4,087,415), it may be transgenic (see for example, U.S. Pat. No. 6,441,145), or recombinant (see for example, U.S. Pat. No. 4,632,981). Heparin may be obtained from pig intestine or bovine lung or it may be obtained from commercial sources. Preferably, the heparin is a “high affinity” heparin enriched for species containing more than one copy of the pentasaccharide. The heparin may have a molecular weight in the range 6,000 to 30,000.

ATH is a covalent complex between antithrombin (AT) and heparin (H), and therefore has a more rapid onset of action than heparin or antithrombin alone. For antithrombin to bind to, and inactivate thrombin, it must first be rendered active through the binding of heparin through a specific pentasaccharide sequence. In the ATH molecule, antithrombin is in the active conformation, ready to bind to and inactivate thrombin, thereby inhibiting clot formation.

ATH has improved potency over heparin because all of the heparin chains in ATH are active. In unfractionated heparin, only 33% of the heparim chains contain a pentasaccharide sequence, (the part of the heparin chain which binds to, and activates antithrombin), while only approximately 1% contain two pentasaccharide sequences. In contrast, in the ATH complex, all the heparin chains contain at least one pentasaccharide sequence, and 25 to 50% of heparin chains contain two pentasaccharide sequences. In addition, unlike heparin, ATH effectively inhibits clot-bound thrombin, which is an important mediator of clot growth.

Conjugates of antithrombin III and heparin (e.g. ATH) allow for administration of lower amounts or dosages of heparin in medical and surgical procedures compared to an amount required when heparin is administered alone.

In particular aspects, the methods, applications and compositions of the invention may utilize:

(a) A covalent conjugate composition comprising glycosaminoglycans linked by covalent linkages to a species comprising at least one primary amino group, wherein said species is directly covalently linked via said amino group to a terminal aldose residue of said glycosaminoglycans, said covalent linkages comprising an alpha-carbonyl amine formed by a substantial amount of subsequent Amadori rearrangement of imines resulting from reaction between said amino group and said terminal aldose residue of said glycosaminoglycans, or a pharmaceutically acceptable salt thereof, wherein said glycosaminoglycans are heparin (H) and said amino-containing species is antithrombin III (AT).

In an embodiment, the covalent linkage comprises a —CO—CH₂—NH— group formed by Amadori rearrangement of a —HCOH—HC═N— group resulting from reaction between the amino group and the Cl carbonyl group of the terminal aldose residue. In another embodiment of a conjugate that may be used in the present invention, the molar ratio of amino-containing species to glycosaminoglycan is less than one. In a further embodiment of a conjugate that can be used in the present invention the linkages comprise an alpha-carbonyl amine formed by essentially complete subsequent Amadori rearrangement.

(b) A covalent conjugate composition comprising glycosaminoglycans and molecules comprising at least one amino group, wherein said amino group is directly linked to said glycosaminoglycans by covalent linkages, wherein said conjugate composition is made by the process comprising:
(i) incubating said glycosaminoglycans with said molecules at a pH and for a time sufficient for imine formation between said amino group and a terminal aldose residue of said glycosaminoglycans, and at a time and temperature sufficient for said imines to undergo a substantial amount of subsequent Amadori rearrangement to an alpha-carbonyl amine forming said covalent linkages; and
(ii) isolating said conjugate composition,
   or a pharmaceutically acceptable salt thereof, wherein said glycosaminoglycans are heparin (H) and said amino-containing molecules are antithrombin III (AT).

In an embodiment of a conjugate that may be used in the present invention, the molar ratio of amino-containing species to glycosaminoglycan is less than one. In another embodiment of a conjugate that may be used in the present invention the imine has undergone essentially complete subsequent Amadori rearrangement, and in a particular embodiment, essentially all of the imines have undergone subsequent Amadori rearrangement. In another embodiment of a conjugate used in the invention the incubation in step (i) is carried out from about 3 days to two weeks at a temperature of 35° C. to 45° C. In another embodiment, of a conjugate used in the invention the incubation in step (i) is carried out for about two weeks, more particularly 10 days.

(c) A conjugate composition comprising a substantial amount of glycosaminoglycans covalently bonded to an amino-containing species by —CO—CH₂—NH—, said CO—CH₂— portion being derived from said glycosaminoglycan and said —NH portion being derived from an amino group of said species, wherein said glycosaminoglycans are heparin (H) and said amino-containing species is antithrombin III (AT).

In an embodiment, the conjugate composition is characterized by one or more of the following: (i) the molar ratio of amino-containing species to glycosaminoglycan is less than one; (ii) the conjugate has a longer half-life than heparin; (iii) it is more effective at inhibiting thrombin than are free ATIII and heparin; (iv) the conjugate inactivates clot-bound thrombin; (v) the molar ratio of heparin to antithrombin is 1:1; (vi) the molecular weight of the conjugate is 69 kD-100 kD; (vii) the conjugate possesses >60%, >90%, >95%, or >98% the antithrombin activity of intact unconjugated heparin; and (viii) essentially all the composition comprises glycosaminoglycans.

(d) A conjugate composition comprising a substantial amount of a complex of the formula: glycosaminoglycan CO—CH₂—NH-protein, wherein the glycosaminoglycan is heparin (H) and the protein is antithrombin III (AT). In an embodiment, the molar ratio of protein to glycosaminoglycan is less than one. In another embodiment, essentially all the composition comprises glycosaminoglycan CO—CH₂—NH-protein.

(e) A covalent conjugate composition comprising glycosaminoglycans linked by covalent linkages to a species comprising at least one primary amino group, wherein said species is directly covalently linked via said amino group to a terminal aldose residue of said glycosaminoglycans, said covalent linkages comprising an amine functional group formed by a substantial amount of reduction of imines resulting from reaction between said amino group and said terminal aldose residue of said glycosaminoglycans, or a pharmaceutically acceptable salt thereof, wherein said glycosaminoglycans are heparin (H) and said amino-containing species is antithrombin III (AT).

In an embodiment, the covalent linkage comprises a —CHR—CH₂—NH— group formed by reduction of an —CHR—HC═N— group resulting from reaction between the amino group and the Cl carbonyl group of the terminal aldose residue. In another embodiment of a conjugate that may be used in the present invention, the molar ratio of amino-containing species to glycosaminoglycan is less than one. In a further embodiment of a conjugate that can be used in the present invention the linkages comprise an amine formed by essentially complete reduction of an imine.

(f) A covalent conjugate composition comprising glycosaminoglycans and molecules comprising at least one amino group, wherein said amino group is directly linked to said glycosaminoglycans by covalent linkages, wherein said conjugate composition is made by the process comprising:
(i) incubating said glycosaminoglycans with said molecules at a pH and for a time sufficient for imine formation between said amino group and a terminal aldose residue of said glycosaminoglycans, and subsequently treating the mixture with a reducing agent capable of reducing the imine function to an amine; and
(ii) isolating said conjugate composition,
   or a pharmaceutically acceptable salt thereof, wherein said glycosaminoglycans are heparin (H) and said amino-containing molecules are antithrombin III (AT).

In an embodiment of a conjugate that may be used in the present invention, the molar ratio of amino-containing species to glycosaminoglycan is less than one. In another embodiment of a conjugate that may be used in the present invention the imine has undergone essentially complete reduction, and in a particular embodiment, essentially all of the imines have undergone subsequent reduction. In another embodiment of a conjugate used in the invention the incubation in step (i) is carried out for about one day at a temperature of 35° C. to 45° C. In another embodiment of a conjugate used in the invention the incubation in step (i) is carried out for about five to 16 hours, more particularly 8 hours.

(g) A covalent conjugate composition comprising glycosaminoglycans and molecules comprising at least one amino group, wherein said amino group is directly linked to said glycosaminoglycans by covalent linkages, wherein said conjugate composition is made by the process comprising:
(i) incubating said glycosaminoglycans with said molecules and a reducing agent at a pH and for a time sufficient for imine formation between said amino group and a terminal aldose residue of said glycosaminoglycans, and in situ reduction of the so formed imine to an amine function; and
(ii) isolating said conjugate composition,
   or a pharmaceutically acceptable salt thereof, wherein said glycosaminoglycans are heparin (H) and said amino-containing molecules are antithrombin III (AT).

In an embodiment of a conjugate that may be used in the present invention, the molar ratio of amino-containing species to glycosaminoglycan is less than one. In another embodiment of a conjugate that may be used in the present invention the imine has undergone essentially complete reduction, and in a particular embodiment, essentially all of the imines have undergone subsequent reduction. In another embodiment of a conjugate used in the invention the linkage reaction is carried out for about one day at a temperature of 35° C. to 45° C. In another embodiment, of a conjugate used in the invention the linkage reaction is carried out for about five to 16 hours, more particularly 8 hours.

(h) A conjugate composition comprising a substantial amount of glycosaminoglycans covalently bonded to an amino-containing species by —CHR—CH₂—NH—, said CHR—CH₂— portion being derived from said glycosaminoglycan and said —NH portion being derived from an amino group of said species, wherein said glycosaminoglycans are heparin (H) and said amino-containing species is antithrombin III (AT).

In an embodiment, the conjugate composition is characterized by one or more of the following: (i) the molar ratio of amino-containing species to glycosaminoglycan is less than one; (ii) the conjugate has a longer half-life than heparin; (iii) it is more effective at inhibiting thrombin than are free ATIII and heparin; (iv) the conjugate inactivates clot-bound thrombin; (v) the molar ratio of heparin to antithrombin is 1:1; (vi) the molecular weight of the conjugate is 69 kD-100 kD; (vii) the conjugate possesses >60%, >90%, >95%, or >98% the antithrombin activity of intact unconjugated heparin; and (viii) essentially all the composition comprises glycosaminoglycans.

A conjugate composition of the invention may be selected that is effective to reduce emboli. In embodiments of the invention a conjugate composition can be selected that results in a 5%, 10%, 15%, 20%, 25%, 30%, 35%, 40%, 50%, 55%, 60%, 65%, 70%, 75%, 80%, or 90% reduction in emboli.

“Emboli” refers to particulate matter found in subjects after medical or surgical procedures which can result in a neurological event. An embolus is generally less than 500 microns in diameter. In an aspect of the invention the emboli are generated from blood elements. In an embodiment of the invention, the emboli are thromboemboli composed of fibrin, platelets, or both. Emboli may be identified using conventional techniques including but not limited to transcranial or transaterial Doppler [where emboli are detected as high-intensity transient signals (HITS)], transesophageal echocardiography, and retinal fluorescein angiography.

The term “therapeutically effective dosage” as used in the present invention refers to a dosage which provides effective prevention or reduction of neurological events or provides protection or preservation of neuronal function for mammals, in particular humans, for the medical conditions and procedures described herein. In an embodiment, a therapeutically effective dosage is an amount effective to prevent or reduce emboli, in particular thromboemboli. As described herein, in general a therapeutically effective dosage in a method or composition of the invention may comprise a dosage ranging between approximately, 0.05 to 100 mg/kg, in particular 0.1 to 50 mg/kg, 0.1 to 20 mg/kg, or 1 to 10 mg/kg, more particularly 2 to 8 mg/kg, most particularly 2 to 6 mg/kg. The glycosaminoglycan and serpin, including conjugates and complexes thereof may be given in 0.1 to 10 mg single intravenous boluses or 0.1 to 1.0 mg/kg intravenous boluses administered at intervals of, for example, every few seconds to several minutes, up to a total dose of 10-20 mg/kg.

A “neurological event” refers to an injury to the central nervous system during or following a medical procedure including but not limited to stroke (focal neurological signs), neurophysiological impairment (subjects are obtunded, sleepy, or delirious) and encephalopathy (abnormalities in thought processes and behaviour). In an aspect of the invention, a neurological event is associated with embolization (embolism), in particular in the cerebral circulation. In another aspect the neurological event is a neurocognitive deficit. In a further embodiment, the neurological event is a change in thought processes and behaviours including but not limited to personality changes, depressions and mood changes.

As described herein the present invention comprises a glycosaminoglycan and a serpin, including complexes and conjugates of same (in particular ATH), and methods for their use. The invention includes therapeutic applications of a glycosaminoglycan and a serpin, including complexes and conjugates of same, as neuroprotective agents. The present invention also provides a method for using a glycosaminoglycan and a serpin, including complexes and conjugates of same, in surgery (e.g. cardiac surgery) which improves neurological outcome.

An aspect of the invention relates to a therapeutic application of a glycosaminoglycan and a serpin, including complexes and conjugates of same, to provide protection to a subject against neurological events, or reduce such neurological events. In an embodiment, the neurological events include but are not limited to neurological events associated with cardiac embolization.

In an aspect the invention provides a method of preventing or reducing neurological events in a subject comprising or consisting essentially of administering a therapeutically effective dosage of a glycosaminoglycan and a serpin, including complexes and conjugates of same, to the subject to prevent or reduce the neurological events.

A glycosaminoglycan and a serpin, including complexes and conjugates of same, can be administered in a therapeutically effective dosage to a subject prior to, during, or after a procedure that may give rise to a neurological event In another aspect, the present invention relates to a therapeutic application of a glycosaminoglycan and a serpin, including complexes and conjugates of same, to provide protection to an individual's brain prior to scheduled, or unscheduled, procedures that may affect the cerebral circulation and/or brain. In an embodiment of the invention, a glycosaminoglycan and a serpin, including complexes and conjugates of same, are administered in a therapeutically effective dosage to a subject prior to, during, or after a procedure that may affect the central nervous system, in particular the brain and/or cerebral circulation.

In another aspect of the invention, a glycosaminoglycan and a serpin, including complexes and conjugates of same, are administered in a therapeutically effective dosage into the circulation or into the brain ventriculocistemal (fluid circulation) system of a subject prior to, during, or after a procedure that may affect the central nervous system, in particular the brain and/or cerebral circulation.

As described herein a glycosaminoglycan and a serpin, including complexes and conjugates of same, can be administered in a therapeutically effective dosage into the circulation or into the brain ventriculocistemal (fluid circulation) system prior to a procedure that may affect the central nervous system, in particular the brain and/or cerebral circulation.

A method of protecting neuronal function in vivo in the central nervous system, in particular the brain and/or the cerebral circulation, is provided comprising the step of administering to a subject a therapeutically effective dosage of a glycosaminoglycan and a serpin, including complexes and conjugates of same, prior to a medical or surgical procedure.

In general, the methods, therapeutic applications, and compositions of the invention may be used with any medical or surgical procedure that may give rise to a neurological event (e.g. emboli in the cerebral circulation) including procedures for coronary artery diseases, valvular heart disease, congenital heart disease, aortic disease, transplantation and a variety of other procedures. Examples of such procedures include but are not limited to surgical procedures, for example cardiopulmonary bypass, cardiac catherization, angioplasty, endarterectomy, and other medical procedures that may affect cerebral circulation. A procedure may also include the administration of pharmaceutical compositions that may affect cerebral circulation. It will be appreciated that the therapeutic applications for the glycosaminoglycan and serpin described herein are by no means limited to the disclosed medical conditions but instead include other conditions that will be apparent to those skilled in the art.

In particular aspects, the methods of the invention can be used to prevent cerebral embolization and to prevent or reduce emboli in the cerebral circulation and/or brain. The methods can be employed on various patients, in particular, those at high risk for cerebral embolization, in order to reduce the risk for cerebral embolization which can lead to neurologic or cognitive complications and death.

In an embodiment, the invention provides a method for reducing emboli (in particular, thromboemboli) in the cerebral circulation in a subject comprising administering an amount of a glycosaminoglycan and a serpin, including complexes and conjugates of same, effective to reduce the emboli.

The invention also relates to a method of cerebral embolic protection in a subject comprising administering an amount of a glycosaminoglycan and a serpin, including complexes and conjugates of same, to reduce emboli in the cerebral circulation.

Further, the invention relates to a method for protecting a subject against cerebral embolization comprising administering an amount of a glycosaminoglycan and a serpin, including complexes and conjugates of same, that reduces the amount of emboli that reach the cerebral vasculature.

The invention also provides methods for eliminating or minimizing cerebral embolization during invasive cardiac procedures.

In a particular aspect of the invention, a glycosaminoglycan and a serpin, including complexes and conjugates of same, are administered to a subject prior to, during, or after a cardiopulmonary bypass procedure. A complication of cardiopulmonary bypass is the formation of emboli that lodge within the cerebral blood vessels resulting in local areas of blood flow cessation or ischemia. In accordance with a method of the invention, administration of a glycosaminoglycan and a serpin, including complexes and conjugates of same, prior to, during or after the bypass procedure can reduce the likelihood of neurological problems. A glycosaminoglycan and a serpin, including complexes and conjugates of same, may be used with other planned surgical procedures where emboli are released into the brain circulation or transient disruption of blood flow to the brain occurs, including but not limited to carotid endarterectomy, clipping of aneurysms, etc.

The invention provides a method of preventing or reducing emboli from a bypassed heart region prior to removal of the region from bypass comprising administering an amount of a glycosaminoglycan and a serpin, including complexes and conjugates of same, effective to prevent or reduce the emboli.

The invention also provides a method for improving the outcome of cardiac surgery in a subject undergoing cardiopulmonary bypass surgery comprising administering a therapeutically effective amount of a glycosaminoglycan and a serpin, including complexes and conjugates of same, effective to prevent or reduce the emboli.

A glycosaminoglycan and a serpin, including complexes and conjugates of same may be administered to a subject during induction of anesthesia, during surgery, and/or after surgery. In embodiments of the invention, administration of a glycosaminoglycan and a serpin is performed after intubation of the patient.

In an aspect of the invention, the glycosaminoglycan and serpin, including conjugates and complexes thereof are administered peri-operatively. In an aspect, the agents are administered pre-sternotomy or post-sternotomy, in particular post-sternotomy. They may be administered in a continuous intravenous infusion, or a plurality of intravenous boluses. They may be administered after intubation but before placing the subject on cardiopulmonary bypass.

The invention also provides a method of performing cardiac surgery, in particular, CABG surgery, in which a therapeutically effective amount of glycosaminoglycan and a serpin, including complexes and conjugates of same, are administered peri-operatively to a subject undergoing cardiopulmonary bypass to reduce the effects of emboli. The glycosaminoglycan and serpin, including complexes and conjugates of same, may be administered during the surgery, particularly after intubation for general anesthesia. They may be administered as a continuous infusion or multiple boluses.

Methods of the invention can additionally comprise administering a heparin antagonist to reverse anticoagulant effects. In an embodiment of the invention the heparin antagonist is protamine sulfate, platelet Factor 4, or heparinases.

In certain aspects of the invention, the glycosaminoglycan and serpin provide synergistic activity in preventing or reducing neurological events. Thus, a method of preventing or reducing cerebral emboli in a patient is provided comprising or consisting essentially of administering to a patient in need thereof, synergistically effective amounts of a glycosaminoglycan and a serpin. By “synergistic activity” or “synergistically effective amount” is meant that a sufficient amount of glycosaminoglycan and serpin will be present in order to achieve a desired result that is greater than the result achieved with each component on its own, e.g. improved reduction of neurological events.

In certain aspects of the invention a glycosaminoglycan and a serpin are administered in combination. In particular, they can be administered concurrently to a patient being treated. When administered in combination, each component may be administered at the same time or sequentially in any order, and at different points in time. Therefore, each component may be administered separately, but sufficiently close in time to provide the desired effect (in particular, a synergistic effect). The components may be associated, for example, they may form a complex or conjugate. In a particular embodiment, the components form ATH.

In embodiments of the invention a heparin or low molecular heparin (e.g. a commercially available heparin or low molecular weight heparin) and antithrombin III (e.g. transgenic or recombinant human antithrombin III) are administered in combination.

The present invention also provides compositions comprising or consisting essentially of a combination of therapeutically effective amounts of glycosaminoglycan and a serpin, including conjugates and complexes thereof, together with a pharmaceutically acceptable excipient, carrier, or vehicle.

In an aspect of the invention a composition is provided comprising or consisting essentially of a heparin or low molecular weight heparin (e.g. a commercially available heparin or low molecular weight heparin) and antithrombin III (e.g. transgenic or recombinant human antithrombin III), together with a pharmaceutically acceptable excipient, carrier, or vehicle

Also contemplated is a pharmaceutical composition in separate containers and intended for simultaneous or sequential administration, comprising a glycosaminoglycan and a serpin, both together with pharmaceutically acceptable excipients, carriers, or vehicles.

In another embodiment, the invention provides a pharmaceutical composition comprising a unit dosage of a glycosaminoglycan, and a unit dosage of a serpin, together with a pharmaceutically acceptable excipient, carrier, or vehicle.

The above mentioned compositions and treatments also include pharmaceutically acceptable salts of the glycosaminoglycan and the serpin, such as sodium, potassium, ammonia, magnesium, and calcium salts.

In accordance with one aspect, a pharmaceutical composition is provided comprising a glycosaminoglycan and a serpin effective to exert a synergistic effect in preventing or reducing neurological events in particular neurological events associated with emboli, more particularly cerebral embolization. The invention also provides pharmaceutical compositions comprising a synergistically effective amount of a combination of a glycosaminoglycan and a serpin in a pharmaceutically acceptable excipient, carrier, or vehicle.

In another aspect the invention relates to a method of using a composition comprising a glycosaminoglycan and a serpin, including complexes and conjugates of same, in the preparation of a medicament for preventing or reducing neurological events, in particular neurological events associated with emboli, more particularly cerebral embolization.

In a further aspect the invention relates to a method of using synergistically effective amounts of a glycosaminoglycan and a serpin in the preparation of a pharmaceutical composition for preventing or reducing neurological events, in particular neurological events associated with emboli, more particularly cerebral embolization.

Since some aspects of the present invention relate to a method of treatment comprising active agents which may be administered separately, the invention also relates to combining separate compositions comprising the active agents in kit form.

The invention also contemplates the use of a composition of the invention or treatment of the invention for preventing, and/or ameliorating disease severity, disease symptoms associated with neurological events, in particular neurological events associated with emboli.

Subjects or patients that may receive a treatment or be administered a composition of the invention include animals, including mammals, and particularly humans. Animals also include domestic animals, including horses, cows, sheep, poultry, fish, pigs, cats, dogs, and zoo animals.

A serpin and a glycosaminoglycan, including conjugates or complexes thereof (e.g. in particular ATH) can be administered by any means that produce contact of an active agent with the agent's sites of action in the body of the patient. The substances can be administered simultaneously or sequentially in any order, and at different points in time, to provide the desired effect. It lies within the capability of a skilled physician or veterinarian to choose a dosing regime that optimizes the effects of the compositions and treatments of the present invention. The compositions may be administered in such oral dosage forms as tablets, capsules (each of which includes sustained release or timed release formulations), pills, powders, granules, elixirs, tinctures, suspensions, syrups, and emulsions. They may also be administered in intravenous (bolus or infusion), intraperitoneal, subcutaneous, or intramuscular form, all using dosage forms well known to those of ordinary skill in the pharmaceutical arts. The compositions of the invention may be administered in intranasal form via topical use of suitable intranasal vehicles, or via transdermal routes, for example using conventional transdermal skin patches. The dosage administration in a transdermal delivery system will be continuous rather than intermittent throughout the dosage regimen.

The dosage regimen will vary depending upon known factors such as the pharmacodynamic characteristics of the particular agent and its mode and route of administration; the species, age, sex, health, medical condition, and weight of the patient, the nature and extent of the symptoms, the kind of concurrent treatment, the frequency of treatment, the route of administration, the renal and hepatic function of the patient, and the desired effect. The effective amount of a drug required to prevent, counter, or arrest progression of a condition can be readily determined by an ordinarily skilled physician or veterinarian.

A composition or treatment of the invention may comprise a unit dosage of a glycosaminoglycan and a serpin. A “unit dosage” refers to a unitary i.e. a single dose which is capable of being administered to a patient, and which may be readily handled and packed, remaining as a physically and chemically stable unit dose comprising either the active agent as such or a mixture of it with solid or liquid pharmaceutical excipients, carriers, or vehicles.

The glycosaminoglycan, serpin, complexes and conjugates thereof, compositions of the present invention or components thereof typically comprise suitable pharmaceutical diluents, excipients, vehicles, or carriers selected based on the intended form of administration, and consistent with conventional pharmaceutical practices. The carriers, vehicles etc. may be adapted to provide a synergistically effective amount of the active components to prevent or reduce neurological events.

Suitable pharmaceutical diluents, excipients, vehicles, and carriers are described in the standard text, Remington's Pharmaceutical Sciences, Mack Publishing Company. By way of example for oral administration in the form of a capsule or tablet, the active components can be combined with an oral, non-toxic pharmaceutically acceptable inert carrier such as lactose, starch, sucrose, methyl cellulose, magnesium stearate, glucose, calcium sulfate, dicalcium phosphate, mannitol, sorbital, and the like. For oral administration in a liquid form, the drug components may be combined with any oral, non-toxic, pharmaceutically acceptable inert carrier such as ethanol, glycerol, water, and the like. Suitable binders (e.g. gelatin, starch, corn sweeteners, natural sugars including glucose; natural and synthetic gums, and waxes), lubricants (e.g. sodium oleate, sodium stearate, magnesium stearate, sodium benzoate, sodium acetate, and sodium chloride), disintegrating agents (e.g. starch, methyl cellulose, agar, bentonite, and xanthan gum), flavoring agents, and coloring agents may also be combined in the compositions or components thereof.

Formulations for parenteral administration of a composition of the invention may include aqueous solutions, syrups, aqueous or oil suspensions and emulsions with edible oil such as cottonseed oil, coconut oil or peanut oil. Dispersing or suspending agents that can be used for aqueous suspensions include synthetic or natural gums, such as tragacanth, alginate, acacia, dextran, sodium carboxymethylcellulose, gelatin, methylcellulose, and polyvinylpyrrolidone.

Compositions for parenteral administration may include sterile aqueous or non-aqueous solvents, such as water, isotonic saline, isotonic glucose solution, buffer solution, or other solvents conveniently used for parenteral administration of therapeutically active agents. A composition intended for parenteral adminstration may also include conventional additives such as stabilizers, buffers, or preservatives, e.g. antioxidants such as methylhydroxybenzoate or similar additives.

A composition or component thereof may be sterilized by, for example, filtration through a bacteria retaining filter, addition of sterilizing agents to the composition, irradiation of the composition, or heating the composition. Alternatively, the active ingredients may be provided as sterile solid preparations e.g. lyophilized powder, which is readily dissolved in sterile solvent immediately prior to use.

In addition to the formulations described previously, the compositions and components thereof can also be formulated as a depot preparation. Such long acting formulations may be administered by implantation (for example, subcutaneously or intramuscularly) or by intramuscular injection. Thus, for example, the agents may be formulated with suitable polymeric or hydrophobic materials (for example, as an emulsion in an acceptable oil), or ion exchange resins, or as sparingly soluble derivatives, for example, as a sparingly soluble salt.

After pharmaceutical compositions have been prepared, they can be placed in an appropriate container and labeled for treatment of an indicated condition. For administration of a composition of the invention, such labeling would include amount, frequency, and method of administration.

The present invention also includes methods of using the compositions of the invention in combination with one or more additional therapeutic agents including without limitation anti-platelet or platelet inhibitory agents such as aspirin, prioxicam, clopidogrel, ticlopidine, or glycoprotein IIb/IIIa receptor antagonists, thrombin inhibitors such as heparin, boropeptides, hirudin, or argatroban; or thrombolytic or fibrinolytic agents, such as plasminogen activators (such as tissue plasminogen activator), anistreplase, urokinase, or streptokinase; or combinations thereof.

The invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

### EXAMPLE 1

Summary

The purpose of this study was to examine the efficacy of an antithrombin-heparin covalent complex (ATH) and equivalent doses of either unfractionated heparin or unfractionated heparin supplemented with transgenic antithrombin in a pig cardiopulmonary bypass (CPB) model.

The test substances were injected at several doses as an iv bolus after sternotomy. About 20 minutes after injection, CPB was begun with hypothermic lowering of the bypass blood temperature to 28° C. Bypass was continued for 2 hours, and normothermia re-established in the last 45 minutes. After bypass, neutralizing protamine sulfate was administered, followed by a 3 hour recovery. Throughout the experiment, microemboli were monitored using arterial ultrasound Doppler HITS (High Intensity Transient Signals) as a primary end point. Activated clotting time (ACT), chest blood accumulation, protein deposition in the bypass circuit, TATs, and D-dimers were monitored as secondary end points.

ATH reduced the HIT rate during CPB to below pre-CPB levels, and the reduction was dose dependant The majority of HITS appeared to represent microemboli, and ATH reduced microemboli formation, particularly at a dose of 3 mg/kg. In all cases, complete anticoagulation reversal was achieved by standard dosing of protamine sulfate.

Methods and Experimental Design

Test System

Pig thrombosis models have been used to evaluate anticoagulants for many years. The effects of anticoagulants in pig models have proven to be predictive of their efficacy in human thrombotic conditions (Munster, Am et al, Comp. Med. 52: 39-43, 2002).

Description of Test System

45, 32-66 kg female Yorkshire pigs were obtained from the University of Guelph Arkell Research Center (Canada) and acclimatized for at least one week prior to the study.

Test Animal Housing

Pigs were housed at the Hamilton Research Center Animal Facility, and animals had ad libitum access to autoclaved Purina Porcine Lab Diet (#5084). The animal room environment and photoperiod was controlled to target conditions of 20° C., 50% humidity & 12 hr light/12 hr dark.

Test Anticoagulants

Heparin of various lots (injection sodium heparin from Organon Teknika Inc.), ATH of various lots Henderson Research Centre and human recombinant antithrombin (AT) of transgenic source (GTC Biotherapeutics) were used.

The Animal Model

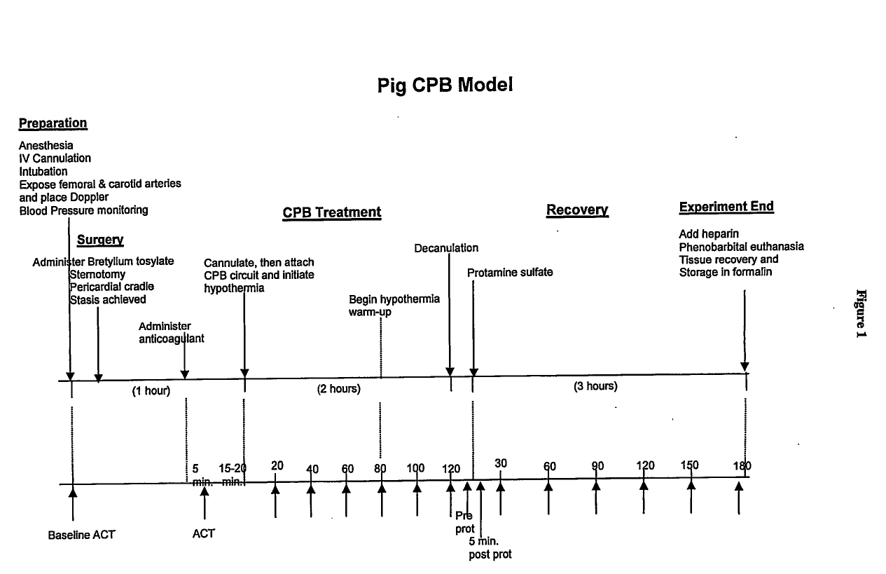

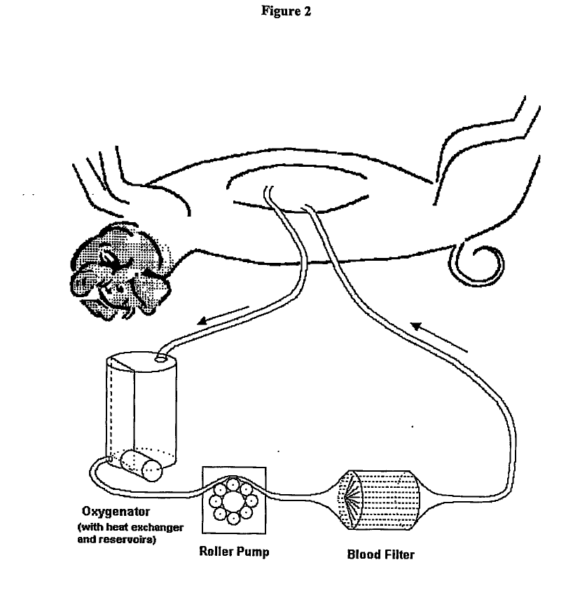

The timelines and procedures for the pig CPB model are shown in FIG. 1 and a diagram of the model is shown in FIG. 2. Anesthesia was initiated by intramuscular injection of ketamine (20 mg/kg), acepromazine (0.2 mg/kg), and atropine (0.05 mg/kg) into Yorkshire pigs and maintained with a mixture of isofluorane (1-3%), oxygen (1.8 L/min), and nitrous oxide (1.2 L/min) delivered through a 9.5F endotrachael tube using a positive pressure respirator. The respiratory rate was adjusted to maintain arterial blood pH, pCO₂, and pO₂ in the physiologic range. During CPB, inhalation anesthesia was delivered through the membrane oxygenator and supplemented with 1-2 ml of iv sodium Phenobarbital. The total anaesthesia time was about 6 hours (1 hour of pre-CPB surgical manipulation, 2 hours of CPB, 3 hours of post-CPB recovery).

A 14 gauge iv cannula was inserted in the marginal auricular vein of the right ear for administration of drugs and fluids, and the femoral artery and both carotid arteries were exposed. A blood pressure transducer was connected to the right femoral artery, while Doppler ultrasound transducer probes were placed on both carotid arteries. “Pre” CPB HIT data were collected, and a “baseline” ACT was measured. A 5 mg/kg bolus of bretylium tosylate was administered intravenously to prevent cardiac arrhythmia, and the heart and great vessels were exposed through a median sternotomy. A pericardial cradle was then created and hemostasis was secured.

The test anticoagulant drug was administered (approximately 50 minutes after taking the “baseline” ACT and more than 20 minutes after sternotomy). Five minutes later, a blood sample was taken for ACT. An ACT of at least 500 seconds was required before proceeding. If this ACT value was not achieved at the beginning or at any time during CPB, the anticoagulant was supplemented with ¼ doses until the ACT exceeded 500 seconds (supplementation of AT+heparin was with heparin only). The ascending aorta was cannulated and connected to the CPB circuit (for “partial bypass”), taking care to avoid air bubbles, and then the right auricle was cannulated to secure a venous line to the CPB circuit. CPB began approximately 20 minutes after injection of the anticoagulant Reduction of core body temperature to 28° C. was initiated and maintained using the CPB circuit heater/cooler unit (the hypothermic state was reached about 20 minutes into CPB).

The CPB circuit was composed of the following components: an affinity integrated CVR membrane oxygenator with a heater/cooler unit, a venous reservoir (maintained at 800 ml with either Ringer's lactate solution containing 7.5% (44 mEq) sodium bicarbonate or reused cavity blood), an on-line arterial blood filter, a roller pump set for 3.5-4 L/min (60% of cardiac output), a 2 stage armored venous drainage catheter, and a return arterial cannula. (See FIG. 2) Mean arterial pressure was maintained above 50 mm Hg. A suction pump was also available, but it was turned off during the bypass to allow manual measurement of chest cavity bleeding. After periodic measurement of cavity blood, the blood was returned to the CPB system via the oxygenator reservoir.

Periodic blood sampling was performed for blood gases, pH analysis, ACT (to set at the beginning of bypass), anticoagulant levels, CBC, hematocit, and TAT levels. Supportive therapy was instituted if needed (epinephrine, dopamine, CaCl₂, Na bicarbonate, etc.), and supplementation of the anticoagulant with ¼ doses was given if the ACT decreased below 500 seconds. Chest blood volume was periodically measured as well as rectal temperature.

After about 1¼ hours of hypothermic CPB, warm up of the pig was begun, and after a total CPB time of 2 hours, the venous CPB line was removed, and remaining blood from the CPB circuit was infused. Decannulation took about 5 minutes, after which a “Pre-Protamine” ACT was taken.

After 5 minutes, anticoagulation was reversed with protamine sulphate to reach the pre-CPB ACT value (50 mg of protamine sulphate, typically used to neutralize 5,000 IU of heparin, was used in this model for 50 Kg pigs given an initial dose of 300 U heparin/Kg, based on previous experience). When stabilization was achieved, the CPB arterial line was removed. Two chest tubes were placed into the pericardial cavity, connected to the chest drainage unit, and kept under negative pressure of 10 ml water. An ACT was then taken (protamine to).

After 3 hours post-CPB (post-protamine), animals were anticoagulated with heparin and then euthanized with sodium phenobarbital. For every pig, the brain, tissue samples from primary soft tissue organs, and a skin sample were saved and stored in 10% buffered formalin.

Selection of Doses

The doses of anticoagulants, were chosen to cover the maximum range that might be encountered in CPB. A heparin dose of 300 units/kg is the equivalent of the usual dose of heparin given to patients undergoing CPB. The dose of heparin yields an ACT over 450 sec. which is the target ACT for most CPB cases. The higher heparin dose (1000 units/kg) was selected to determine whether supratherapeutic doses of heparin would provide better reduction in HITS and/or microthrombi than the usual heparin doses.

Animal Identification and Treatment Groups

A total of 45 female pigs, individually identified by numbered ear tags following arrival, were assigned to 8 Groups. Pigs were dosed with the concentrations of anticoagulants as specified in Table 1. Doses shown on the graphs in the Figures are the initial doses (not final doses when supplementation was required). Four 6 mg/kg ATH-dosed pigs and one 6 mg/kg AT+300 U/kg heparin-dosed pig had to be eliminated from the study due to complications of urticaria and splanchnic pooling that could interfere with the efficacy assessments of this study.

Observations

Doppler HITS

Trans-arterial ultrasound Doppler HITS were measured by placing two round 2 MHz probes (Spencer Technologies) liberally covered with Aquasonic 100 ultrasound transmission gel (Parker Labs) on each of the two carotid arteries. Care was taken to avoid air bubbles in the gel, and the probes were oriented at a 20-degree angle with respect to the artery. Adjustments were made to optimize the signal, which was sent to a computer in the TCD 2020 transcranial Doppler machine (Nicolet Vascular) for digitized storage and computer recognition of HITS.

Discrimination of microemboli from micro air bubbles and dislodged fat was investigated in a preliminary study, and distinctive patterns characteristic of each agent were seen. In this study, this level of discrimination was not needed, so all HITS were counted.

HITS were integrated for segments of time represented by the blood sampling times shown on the time-line (FIG. 1). These integrated values were then analyzed by three methods. Total HITS were summed for the study segments “Pre-CPB”, “CPB”, and “Post-CPB”, an average hit rate (normalized per hour) was determined for each of these study segments, and normalized hit rates were determined for smaller sampling segments (to give a more dynamic picture of HIT response).

Blood Loss Measurements

Chest cavity blood was collected periodically, measured for volume, and returned to the CPB circulation.

Protein Deposition Measurements

At the end of bypass, the filter/oxygenator/reservoir linked units were flushed with Ringer's solution, followed by 2 liters of 2M sodium hydroxide perfused in the CPB circuit for 1 hour. The sodium hydroxide wash was sampled and analyzed for total protein and hemoglobin. For hemoglobin, samples were diluted 1:10, measured at 540 nm on a spectrophotometer, and the values compared to a standard curve. For protein, samples were diluted 1:100 and measured with a standard commercial protein assay.

Blood Samples for Coagulation Analysis and Reference

Periodic blood samples were taken from the pig as indicated on the timeline (FIG. 1) and as needed 2 ml EDTA samples were taken for CBC, 5 ml citrate plasma samples for anticoagulant assays/TATs & D-dimer, 3 ml samples for ACT, 1 ml samples in pre-heparinized syringes for blood gas/pH/sodium/potassium.

Tissue Analysis

The brain, tissue samples from primary organs and a skin sample were saved and stored in formalin for every pig for potential future processing.

Statistical Analysis

Data means and standard errors of the mean (SEM) (derived from population standard deviations) were calculated for graphical representation.

Results

Doppler HITS/HR

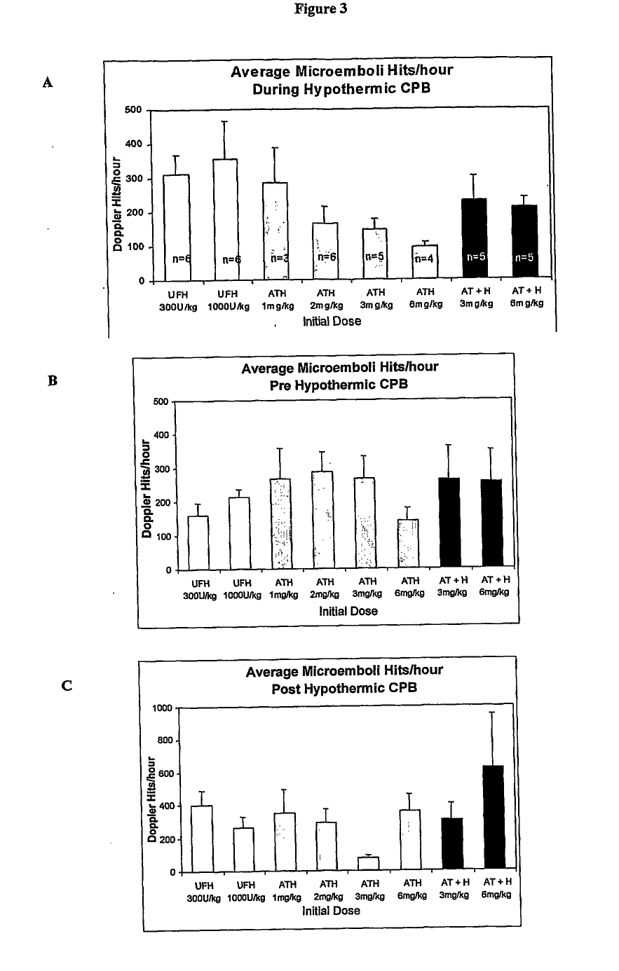

Post-sternotomy, pre-CPB HITS occurred at a rate of about 160-300/hr regardless of the anticoagulant given or its dose (FIG. 3B). Since anticoagulants were injected well after sternotomy, it is likely that pre-CPB HITS reflect an average of background activation of the coagulation cascade as a result of tissue damage induced by sternotomy and the initial effects of drug on this background. This sensitivity to drug effects is expected to be low during this period.

During CPB, HITS increased for UFH by almost 50% (FIG. 3A), regardless of dose. A dose-dependent decrease in CPB HITS was seen with ATH at doses ranging from 1-6 mg/kg. AT (3 mg/kg)+H (300 U/kg) resulted in a HITS rate between UFH and the 2 mg/kg ATH. Doubling the AT dose did not significantly change the HITS rate. ATH 3 mg/kg was the only treatment with a post-CPB HITS rate less that the rate pre-CPB (FIG. 3C).

Bleeding

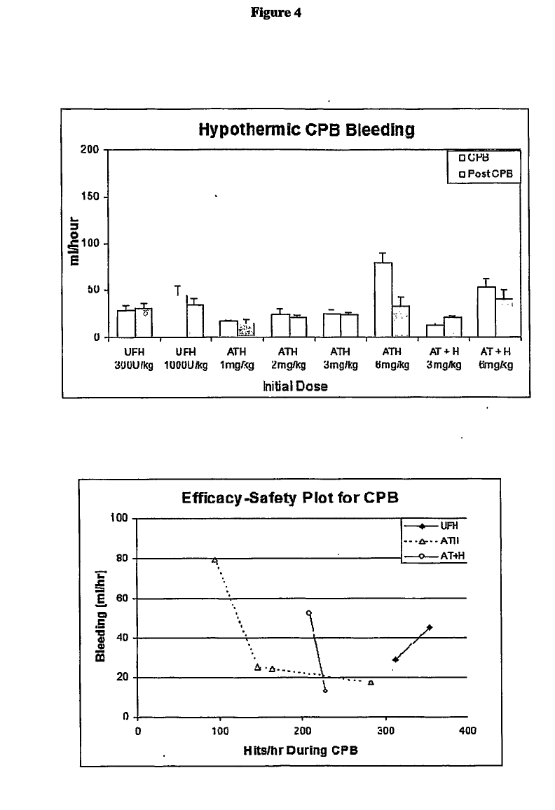

Bleeding (FIG. 4 expressed as ml/hr) was low for all animals in this study. Therefore, all tested anticoagulants show comparable safety profiles during CPB. When the data are replotted as an efficacy-safety plot (using HIT rate for efficacy), ATH has a better efficacy-safety profile than either AT+heparin or heparin alone. It may be of interest that the profile slope of UFH (and of an another heparin from a different study, data not shown) is positive, while the slopes of both AT and ATH are negative.

Protein Deposition on the CPB Circuit

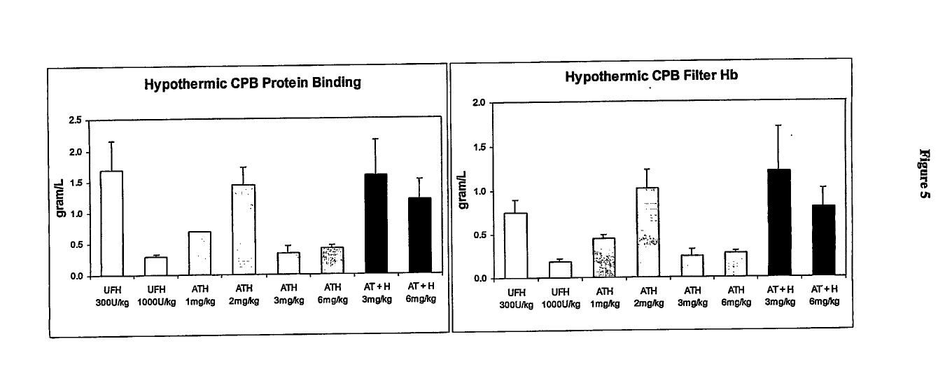

Protein Deposition on the CPB circuit, measured either as total protein or as hemoglobin, is shown in FIG. 5. Protein deposition was a pooled measurement of the sodium hydroxide wash of the blood reservoir (exposed to static and low flow rate blood), oxygenator, and blood filter (exposed to high flow rate blood). UHF (1000 U/kg) and 3 and 6 mg/kg ATH resulted in little protein and fibrin accretion on the circuit, whereas 300 U/kg UFH, 2 mg/kg ATH, and AT+H are less effective at reducing deposition.

Activated Clotting Time (ACT)

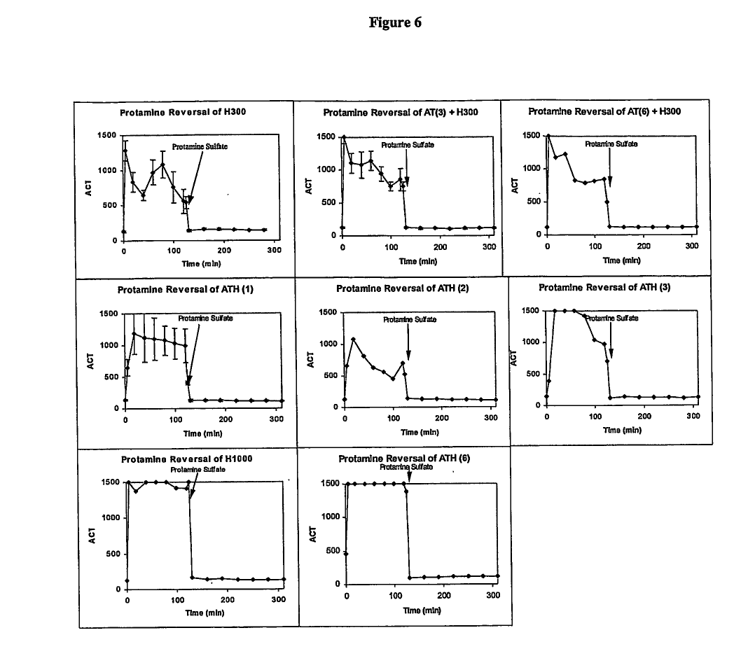

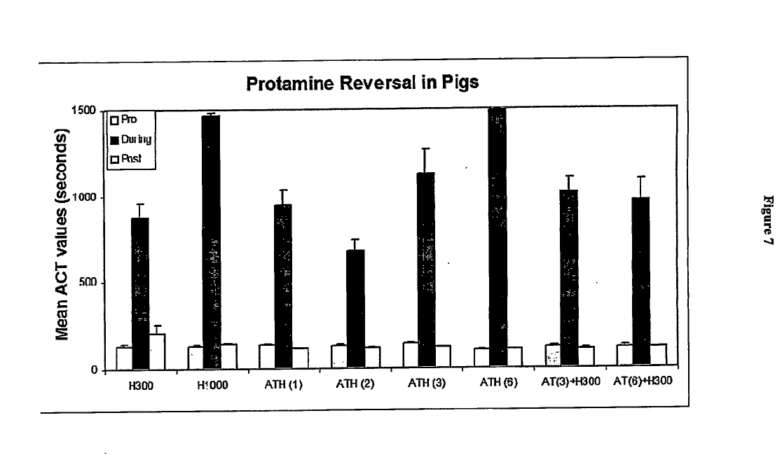

The anticoagulant effects and protamine sulfate reversibility of the test agents were demonstrated in the group averaged dynamic representation of the ACT data (FIG. 6. The highest mean ACT levels during CPB (also requiring the least amounts of supplementation) were achieved with 1000 U/kg UFH, 3 mg/kg ATH and 6 mg/kg ATH. The AT+H profile was midway between the profiles for 300 U/kg heparin and 1 mg/kg ATH. Protamine sulphate neutralized the anticoagulant effect of all test articles, bringing the ACT back to baseline levels. Heparin supplementation was required for both AT+H doses. The protamine sulfate reversal effects on all test agents are summarized in FIG. 7.

TATs

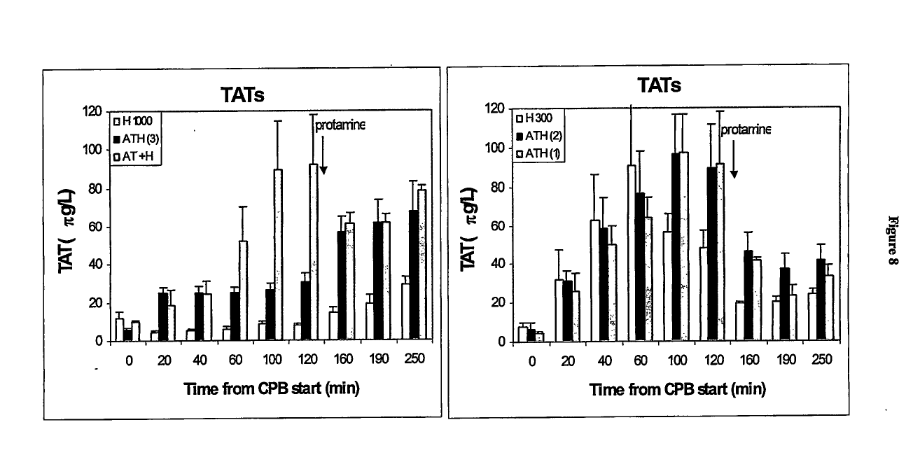

Thrombin antithrombin complexes (TAT) (FIG. 8) increase in a characteristic way during CPB and decline thereafter. It is not clear from these data whether the decline is dependent on either the termination of CPB, the neutralization of anticoagulant by protamine sulfate (not likely), or simply the time after initiation of CPB. The very low level of TATs and flat profiles during CPB (as well as the gradual increase following CPB) for 1000 U/kg UFH and 3 mg/kg ATH are in striking contrast to the rapid rise in TATs for AT+H and lower doses of ATH and UPH. It is also not clear from these data whether the gradual increase in TATs following CPB for 1000 U/kg UFH and 3 mg/kg ATH is due to protamine reversal or just coincidental.

D-Dimers

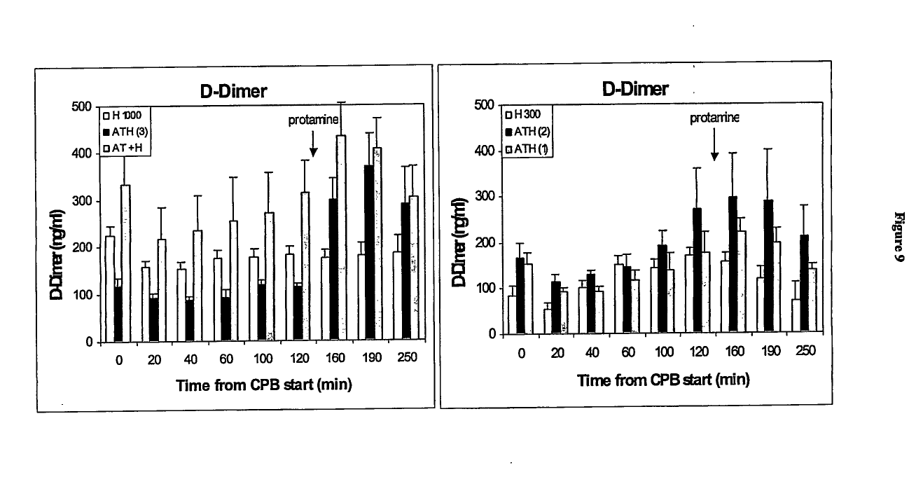

D-dimer (FIG. 9) levels are a less sensitive index of thrombin activation than TATs. There is no evidence of an increase in D-dimer level during CPB for 1000 U/kg UFH or 3 mg/kg ATH, though there is a trend toward an increase for all other agents. Protamine reversal causes a pronounced increase in the 3 mg/kg ATH D-dimer level but does not affect the level for 1000 U/kg UFH.

Conclusions

1. The dose-dependent CPB HITS rate decreased in response to injection of anticoagulants. Thus, the majority of HITS represent microemboli.
2. ATH reduced the HITS rate during CPB. During CPB, UFH yields a HITS rate almost twice that seen pre-CPB, even for heparin doses as high as 1000 U/kg. ATH at a dose of 3 mg/kg reduces CPB HITS rate to about half the pre-CPB rate. ATH at a dose of 6 mg/kg reduced the CPB HITS rate further, and also appeared to reduce the pre-CPB rate. AT+H (AT dose 3 mg/kg) also affects the CPB HITS rate compared with the pre-CPB HITS rate. Protein accretion in the bypass circuit tended to confirm the efficacy of ATH and showed an equivalent lowering of protein accretion as that produced by 1000 U/kg heparin.
3. All anticoagulant agents tested can be completely reversed with standard doses of protamine sulfate.
4. HITS occurred pre-sternotomy and were reversible with protamine indicating that any tissue insult has the potential to create embolization. This supports broad applications of the technology described herein in conditions or procedures involving tissue insult which results in embolization.

5. Table 3 below shows the difference in heparin concentration (in units) when heparin alone is given for CPB versus the heparin in ATH. Less heparin is given in ATH. Thus, linking heparin and AT results in a product with an unexpected advantage having greater in vivo activity compared with heparin alone.

TABLE 3

| | mg AT/Kg | mg Heparin/Kg |
|---|---|---|
| H300 | — | 1.88 |
| ATH 3 | 3.00 | 0.92 |
| ATH 6 | 6.00 | 1.83 |

### EXAMPLE 2

Segments of brains from pigs that had undergone CPB with either heparin or ATH anticoagulation were embedded in paraffin, sectioned, and stained with MSB. Fibrin thrombi in the microvasculature were quantified, and the number of microthrombi compared with the number of HITS determined by carotid ultrasound. For this comparison, the total number of HITS during CPB (the only variable influenced by anticoagulation) was used.

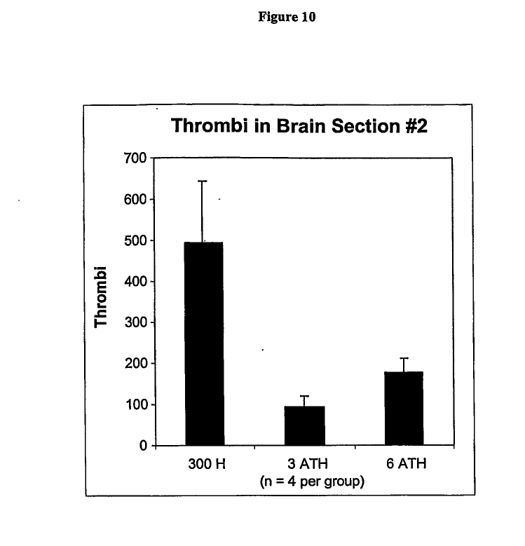

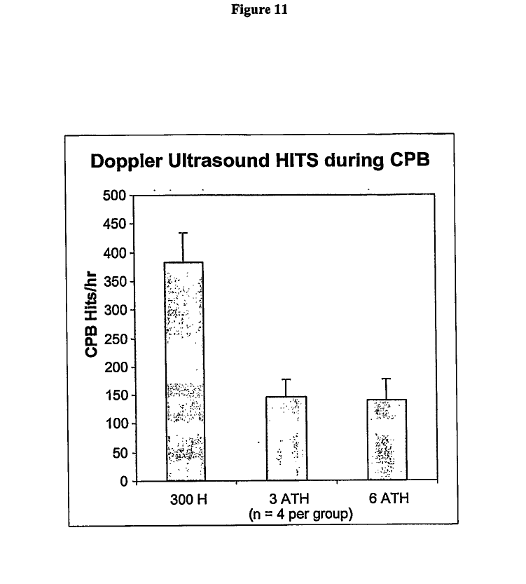

Initial analysis focused on a coronal section (section 2 out of a total of 8) from the brains of three groups of pigs (300 U/kg heparin, 3 mg/kg ATH, or 6 mg/kg ATH). Fibrin-thrombi in both the right and left cerebral hemispheres were counted and the results combined. The results are illustrated in Table 2 and FIGS. 10 and 11.

Based on these results, pigs given ATH during CPB had fewer thrombi (FIG. 10) and HITS (FIG. 11) than those given heparin.

TABLE 1
Treatment Groups

| | | | | Initial | Initial | Total | Total | Total | Neutralizing |
|---|---|---|---|---|---|---|---|---|---|
| | | | | heparin | heparin | heparin | heparin | AT | Prot. |
| | | | Animals | mass | activity | mass | activity | mass | Sulfate |
| | | | per | equiv. | equiv. | equiv. | equiv. | equiv. | given |
| Group | Anticoagulant | | roup | (mg/kg) | (U/kg) | (mg/kg) | (U/kg) | (mg/kg) | (mg/kg) |
| 1 | 300 | U/kg heparin | 6 | 1.9 | 300 | 2.6 | 421 | — | 0.52 |
| 2 | 1000 | U/kg heparin | 6 | 6.3 | 1000 | 6.3 | 1000 | — | 1.94 |
| 3 | 1 | mg/kg ATH | 3 | 0.4 | 225 | 0.6 | 393 | 2.0 | 1.18 |
| 4 | 2 | mg/kg ATH | 6 | 0.6 | 385 | 0.8 | 512 | 2.7 | 1.06 |
| 5 | 3 | mg/kg ATH | 5 | 1.1 | 677 | 1.1 | 707 | 3.6 | 1.59 |
| 6 | 6 | mg/kg ATH | 8 | 1.9 | 1217 | 1.9 | 1217 | 6.0 | 1.29 |

| | | | | | | | | |
|---|---|---|---|---|---|---|---|---|
| 7 | 3 mg/Kg AT + | 5 | 1.9 | 300 | 2.2 | 345 | 3.0 | 1.06 |
| | 300 U/kg heparin | | | | | | | |
| 8 | 6 mg/kg AT + | 6 | 1.9 | 300 | 2.7 | 420 | 6.0 | 1.05 |
| | 300 U/kg heparin | | | | | | | |

TABLE 2

| | | Fibrin emboli | | | CPB | Total |
|---|---|---|---|---|---|---|
| Group | Pig | left | right | total | Hits/hr | Hits |
| 300 H | 22 | 383 | 417 | 800 | 243 | 1217 |
| | 23 | 238 | 103 | 341 | 530 | 1723 |
| | 25 | 57 | 25 | 82 | 356 | 2128 |
| | 59 | 406 | 346 | 752 | 403 | 2796 |
| | 98 | 8 | | | 215 | 2861 |
| 3 ATH | 79 | 24 | 8 | 32 | 143 | 785 |
| | 81 | 66 | 89 | 155 | 230 | 1171 |
| | 89 | 77 | 11 | 88 | 219 | 803 |
| | 91 | 19 | 9 | 28 | 65 | 654 |
| | 92 | 67 | 99 | 166 | 76 | 681 |
| 6 ATH | 102 | 22 | 43 | 65 | 262 | 2721 |
| | 103 | 95 | 102 | 197 | 144 | 3068 |
| | 104 | 103 | 113 | 216 | 74 | 1745 |
| | 107 | 83 | 148 | 231 | 76 | 277 |
| | 108 | 447 | | | 90 | 2109 |

Means SEM

| | Fibrin emboli | | CPB hits/hr | | Total Hits | |
|---|---|---|---|---|---|---|
| 300 H | 493.8 | 148.6 | 383.0 | 51.4 | 1966.0 | 288.9 |
| 3 ATH | 93.8 | 26.2 | 146.6 | 30.9 | 818.8 | 82.8 |
| 6 ATH | 177.3 | 33.0 | 139.0 | 38.2 | 1952.8 | 541.1 |

### FULL CITATIONS FOR REFERENCES REFERRED TO IN THE SPECIFICATION

1. Gugino L D, Chabot R J, Aglio L S, et al. QEEG changes during cardiopulmonary bypass: relationship to postoperative neuropsychological function. Clin Electroencephalogr, 30(2), 53-63, 1999.
2. Vingerhoets G, Van Nooten G, Vermassen F, De Soete G, Jannes C. Short-term and long-term neuropsychological consequences of cardiac surgery with extracorporeal circulation. Eur J Cardiothorac Surg, 11(3), 424-31, 1997.
3. Rodig G, Rak A, Kasprzak P, et al. Evaluation of self-reported failures in cognitive function after cardiac and noncardiac surgery. Anaesthesia, 54(9), 826-30, 1999.
4. Newman M F, Grocott H P, Mathew J P, et al. Report of the substudy assessing the impact of neurocognitive function on quality of life 5 years after cardiac surgery. Stroke, 32(12), 2874-81, 2001.
5. Grigore A M, Grocott H P, Mathew J P et al. The rewarming rate and increased peak temperature alter neurocognitive outcome after cardiac surgery. Anesth Analg, 94(1), 4-10, 2002.
6. Llinas R, Barbut D, Caplan L R Neurologic complications of cardiac surgery. Prog Cardiovasc Dis, 43(2), 101-12, 2000.
7. Di Carlo A, Perna A M, Pantoni L, et al. Clinically relevant cognitive impairment after cardiac surgery: a 6-month follow-up study. J Neurol Sci, 188(1-2), 85-93, 2001.
8. Jacobs A, Neveling M, Horst M, et al. Alterations of neuropsychological function and cerebral glucose metabolism after cardiac surgery are not related only to intraoperative microembolic events. Stroke, 29(3), 660-7, 1998.
9. Van Dijk D, Jansen E W, Hijman R et al. Cognitive outcome after off-pump and on-pump coronary artery bypass graft surgery: a randomized trial. JAMA, 287(11), 1405-12, 2002.
10. McMaster University, Animal Utilization Protocol, # 00-05-15, “Modification of Cerebral Microemboli During Standard Cardiopulmonary Bypass in a Pig Model.”
11. Munster A M, Olsen A K, Bladbjerg E M. Usefulness of human coagulation and fibrinolysis assays in domestic pigs. Comp Med. 52: 39-43, 2002.
12. Hogg P J, Jackson C M. Heparin promotes the binding of thrombin to fibrin polymer. J Biol. Chem. 265: 241-47, 1990.
13. Weitz J I, Leslie B, Hudoba M. Thrombin binds to soluble fibrin degradation products where it is protected from inhibition by heparin-antithrombin. Circulation, 97: 544-52, 1998.
14. Xiao Z, Theroux P. Platelet activation with unfractionated heparin at therapeutic concentrations. Circulation, 97: 251-56, 1998.
15. Moller J T, Cluitmans P, Rasmussen L S, et al. Long-term postoperative cognitive dysfunction in the elderly ISPOCD1 study (International Study of Post-Operative Cognitive Dysfunction). Lancet, 351: 857-861 (1998)
16. Weitz, J I et al J Clin Invest. 86:385-91, 1990

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. All publications, patents and patent applications mentioned herein are incorporated herein by reference for the purpose of describing and disclosing the domains, cell lines, vectors, methodologies etc. which are reported therein which might be used in connection with the invention. Nothing herein is to be construed as an admission that the invention is not entitled to antedate such disclosure by virtue of prior invention.

It must be noted that as used herein and in the appended claims, the singular forms “a”, “an”, and “the” include plural reference unless the context clearly dictates otherwise. Thus, for example, reference to “a host cell” includes a plurality of such host cells, reference to the “antibody” is a reference to one or more antibodies and equivalents thereof known to those skilled in the art, and so forth.

Below full citations are set out for the references referred to in the specification.

We claim:

1. A method of preventing or reducing neurological events in a subject comprising administering a therapeutically effective dosage of a glycosaminoglycan and a serpin to the subject to prevent or reduce the neurological events.

2. A method of claim 1 wherein the neurological event is associated with emboli.

3. A method of any preceding claim wherein the emboli are thromboemboli.

4. A method of any preceding claim wherein the serpin is antithrombin III.

5. A method of claim 4 wherein the antithrombin III is transgenic antithrombin III.

6. A method of any preceding claim wherein the glycosylaminoglycan is heparin or low molecular weight heparin.

7. A method of any preceding claim wherein the glycosylaminoglycan and serpin form a complex or conjugate.

8. A method of claim 7 wherein the complex or conjugate comprises antithrombin III covalently linked to heparin.

9. A method of claim 8 wherein the complex or conjugate comprises ATH.

10. A method of any preceding claim wherein the glycosaminoglycan and the serpin are administered prior to, during, or after a procedure that may give rise to a neurological event.

11. A method of claim 10 wherein the procedure affects the central nervous system.

12. A method of claim 11 wherein the procedure affects the brain and/or cerebral circulation.

13. A method of claim 10 wherein the procedure is a surgical procedure.

14. A method of claim 13 wherein the procedure is cardiopulmonary bypass, cardiac catherization, angioplasty, or endarterectomy.

15. A method as claimed in claim 2 comprising reducing emboli in the cerebral circulation in a subject comprising administering an amount of a glycosaminoglycan and a serpin effective to reduce the emboli.

16. A method of any preceding claim for protecting a subject against cerebral embolization comprising administering an amount of a glycosaminoglycan and a serpin that reduces the amount of emboli that reach the cerebral vasculature.

17. A method of performing cardiac surgery comprising administering a therapeutically effective amount of a glycosaminoglycan and a serpin peri-operatively to a subject undergoing cardiopulmonary bypass to prevent or reduce the effects of emboli.

18. A method of claim 2 comprising preventing or reducing emboli from a bypassed heart region prior to removal of the region from bypass by administering an amount of a glycosaminoglycan and a serpin effective to prevent or reduce the emboli.

19. A method of using a glycosaminoglycan and a serpin in the preparation of a medicament for the prevention or inhibition of cerebral embolization.

20. A method of using synergistically effective amounts of a glycosaminoglycan and a serpin in the preparation of a pharmaceutical composition for preventing or reducing neurological events.

21. A method of any of claims 17 to 20 wherein the glycosylaminoglycan and serpin form a complex or conjugate.

22. A method of claim 21 wherein the complex or conjugate comprises antithrombin III covalently linked to heparin.

23. A method of claim 21 wherein the complex or conjugate comprises ATH.

24. A pharmaceutical composition comprising a combination of a glycosaminoglycan and a serpin effective to exert a synergistic effect in preventing or reducing neurological events.

25. A pharmaceutical composition comprising a combination of a heparin or low molecular weight heparin and antithrombin III effective to in preventing or reducing neurological events.
